(19) United States
(12) Patent Application Publication
(10) Pub. No.: US 2026/0165884 A1
(43) Pub. Date: Jun. 18, 2026

(54) AUTO-DARKENING LASER LIGHT FILTER AND WELDING HELMET

(71) Applicant: TECMEN ELECTRONICS CO., LTD, Nanjing (CN)

(72) Inventor: Ziqian WU, Nanjing (CN)

(21) Appl. No.: 19/531,755

(22) Filed: Feb. 6, 2026

Related U.S. Application Data
(63) Continuation of application No. PCT/CN2024/091424, filed on May 7, 2024.

(30) Foreign Application Priority Data
Apr. 7, 2024 (WO) PCT/CN2024/086414

(51) Int. Cl. A61F 9/06

(52) U.S. Cl. CPC A61F 9/067

(57) ABSTRACT

An auto-darkening laser light filter for welding and a welding helmet including the same. The welding helmet includes a metal mask having an opening and an auto-darkening laser filter positioned behind the opening. The auto-darkening laser filter includes a metal panel comprising an aperture, an auto-darkening filter positioned behind the aperture, and a laser lens configured to filter a range of wavelengths of light. The metal panel is positioned on an exterior surface of the auto-darkening laser filter and adjacent to the metal mask. The laser lens positioned on the auto-darkening filter.

### CROSS-REFERENCE TO RELATED AND CLAIM OF PRIORITY

This application is a Bypass Continuation of International Application No. PCT/CN2024/091424, filed May 7, 2024, which claims priority to International Application No. PCT/CN2024/086414, filed Apr. 7, 2024, the disclosures of which are herein incorporated by reference in their entirety.

### TECHNICAL FIELD

The present disclosure relates generally to welding helmets and, more specifically, relates to an auto-darkening laser filter for a welding helmet.

### BACKGROUND

In a welding operation, an operator wears a welding helmet, or hood, on his/her head to prevent him/her from being hurt by various harmful rays caused by welding-arc ignition and any matter caused by welding. Some welding helmets are provided with automatic darkening filters composed of a liquid crystal display, a control circuit, a solar panel or battery, a photo-sensitive sensor, and light filters. The light filters prevent harmful ultraviolet and infrared light emitted by welding equipment from reaching and harming a welder's eyes while allowing in sufficient light for the welder to safely see his or her work. However, current welding helmets and auto darkening filters are only made for protect against harmful light emitted by traditional welding methods.

### SUMMARY

This disclosure relates to an auto-darkening laser light filter for welding and a welding helmet including the same.

In an embodiment, a welding helmet is provided. The welding helmet includes a metal mask having an opening and an auto-darkening laser filter positioned behind the opening. The auto-darkening laser filter includes a metal panel comprising an aperture, an auto-darkening filter positioned behind the aperture, and a laser lens configured to filter a range of wavelengths of light. The metal panel is positioned on an exterior surface of the auto-darkening laser filter and adjacent to the metal mask. The laser lens positioned on the auto-darkening filter.

In another embodiment, an apparatus for filtering light is provided. The apparatus includes a metal panel comprising an aperture, an auto-darkening filter configured to selectively filter light, and a laser lens configured to filter a range of wavelengths of light. The metal panel is positioned on an exterior surface of the apparatus. The auto-darkening filter is positioned behind the aperture. The laser lens is positioned on the auto-darkening filter.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication or connection between two or more elements, whether or not those elements are in physical contact with one another. The terms “include” and “comprise,” as well as derivatives thereof, mean inclusion without limitation. As used herein, the terms “faceward,” “outer face,” “front face,” or “front side” refer to a face or side towards outside and departing from the face of an operator when the welding helmet has been worn on his/her head; and the terms “inner face” “back face,” or “back side” refer to a face or side towards inside and to the face of the operator when the welding helmet has been worn on his/her head.

The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. As used herein, when referring to a range or number, “about” means at least within % 10 and any range disclosed is inclusive of the start and end of the range (e.g., a range from 1 to 10 includes 1 and 10) unless specified otherwise.

The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIGS. 1A and 1B illustrate views of a welding helmet with an auto-darkening laser filter being worn by a user according to embodiments of the present disclosure;

FIG. 2 illustrates an exploded view of the welding helmet with the auto-darkening laser filter of FIGS. 1A and 1B according to embodiments of the present disclosure;

FIG. 3 illustrates an exploded view of an auto-darkening laser filter according to embodiments of the present disclosure; and

FIG. 4 illustrates a diagram of an auto-darkening laser filter selectively filtering light emitted towards a user's eye according to embodiments of the present disclosure.

### DETAILED DESCRIPTION

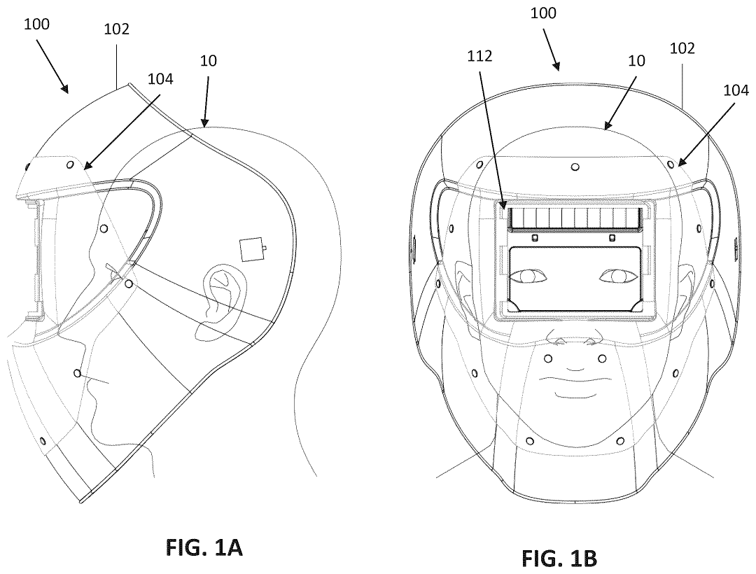

FIGS. 1A through 4, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably-arranged system or device.

New laser welding systems are an improvement over traditional welding systems, such as metal inert gas (MIG) welding, tungsten inert gas (TIG) welding systems, or resistance spot welding, by offering significantly higher precision (e.g., four times more precise) and closer control of the energy applied to materials to be welded together. Laser welding thus also causes less stress in the materials. Furthermore, laser welding is also significantly faster, providing up to four or five times more distance welded per minute. To facilitate such advantageous welding, a laser operating in the infrared and ultraviolet light spectrums, spectrums that are harmful to the human eyes and skin, energizes the materials to be bonded and melting the material to form a weld. Typically, welding lasers operate between and including 1060 nanometer (nm) and 1080 (nm) wavelengths, although other wavelengths may also be used. As this disclosure recognizes, traditional welding protection does not meet the safety needs of laser welding.

FIGS. 1A and 1B illustrate views of a welding helmet 100 with an auto-darkening laser filter (ADLF) 112 being worn by a user 10 according to embodiments of the present disclosure. The embodiment of the welding helmet 100 shown in FIGS. 1A and 1B is for illustration only. Other embodiments of the welding helmet 100 could be used without departing from the scope of the present disclosure.

As depicted in FIGS. 1A and 1B, a welding helmet 100 includes first and second masks 102 and 104 as well as an ADLF 112 disposed therein. FIG. 1A is a side view of the welding helmet 100 and FIG. 1B a front view of the welding helmet 100 when worn by the user 10. FIGS. 1A and 1B provide a transparent or see through view of the user 10 wearing the welding helmet 100 for the purpose of illustrating the position of the head and eyes of the user 10 while wearing the welding helmet 100. However, as described in greater detail below, in various embodiments, various components of the welding helmet 100 are made of opaque materials such that the head and/or eyes of the user 10 are not visible beneath the welding helmet 100 when worn.

The welding helmet 100 with the ADLF 112 of this disclosure provides safety equipment while also being adaptable to different welding environments, that is, for use for different welding equipment whether it be a different laser welding system or a traditional (e.g., MIG or TIG) welding system. When worn by a user 10, the welding helmet 100 is configured to cover the user's face to inhibit particulate matter from harming the person's face. The ADLF 112 is configured to inhibit potentially harmful electromagnetic radiation (light) from reaching the person's eyes, as will be described in more detail below. As described in greater detail below, other components of the welding helmet 100, such as the first and second masks 102 and 104 prevent or inhibit the potentially harmful electromagnetic radiation from reaching other portion of the person's face.

As described in greater detail below, the ADLF 112 is configured to darken the light (i.e., lower the intensity) of the light transmitted into the welding helmet 100. The ADLF 112 may be configured to transition from shade 1 through shade 15 of a standard governing body shade scale (e.g., as established by ANSI or OSHA). A shade number indicates the intensity of light that is allowed to pass through a filter to a person's eyes. Generally, the lower numbered shades allow more light and the higher number shades filter more light for more darkness and a diminishing of the intensity of transmitted light.

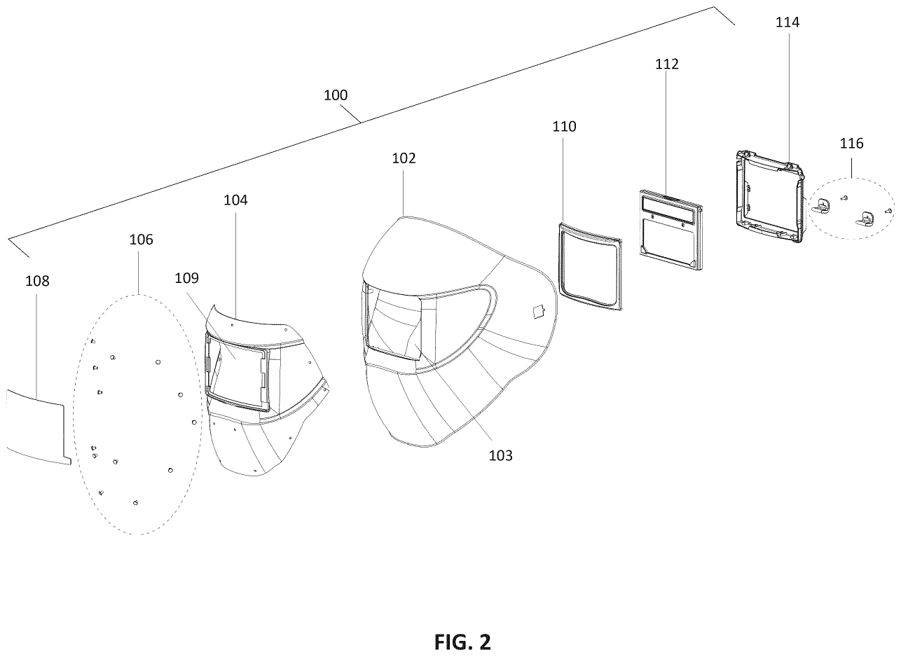

FIG. 2 illustrates an exploded view of the welding helmet 100 including the ADLF 112 according to various embodiments of the present disclosure. The embodiment of the welding helmet 100 shown in FIG. 2 is for illustration only. Other embodiments of the welding helmet 100 could be used without departing from the scope of the present disclosure.

As illustrated in FIG. 2, the welding helmet 100 comprises the first mask 102, the second mask 104, a cover lens 108, and the ADLF 112. The second mask 104 is coupled to the first mask 102 via fasteners 106. The fasteners 106 may be rivets, screws, or other mechanical fasteners known by those of ordinary skill in the art or the fasteners may be an adhesive, magnet, or other permanent or temporary bonding means. The second mask 104 is coupled to an outer face of the first mask 102, sealing the first mask 102 such that light passes only through a window or opening 103 of the first mask 102 and then a window 109 of the second mask 104. The second mask 104 may be coupled to the first mask 102 so that no light, or an insubstantial and thus unharmful amount of light, may be passed between the first mask 102 and the second mask 104, except through the window or opening 103 and the window 109 as mentioned.

In various embodiments, the first mask 102 may be made from a non-metallic material such as a polymer or plastic material capable of blocking, absorbing, and/or dissipating harmful electromagnetic radiation. The second mask 104 may be made from a material that blocks, absorbs, and/or dissipates laser light electromagnetic radiation. For example, the second mask 104 may be made from a metal, including aluminum and aluminum alloys, other lightweight metals, such as titanium or heavier metals such as iron, steel, or copper. In various embodiments, the second mask 104 may be removable for instances where laser welding is not used.

A headband structure (not shown) may be operatively coupled to the first mask 102 to enable a user to wear and adjust the fit of the welding helmet 100 to the face and/or head of a user 10. In other embodiments, not shown, the first mask 102 may include back and top portions to surround the head of user.

The cover lens 108 is installed on the window 109 of the second mask 104. The cover lens 108 is configured to inhibit spatter, sparks, or particulates caused by welding from passing through the window 109 to protect both the ADLF 112 and a person wearing the welding helmet 100. The cover lens 108 is configured to be removed and replaced when the cover lens 108 reaches the end of its service life (e.g., when the cover lens has too many scratches, dents, or a change in clarity as determined by a user). The cover lens 108 may be made out of any suitable transparent material including glass or plastic.

The ADLF 112 is coupled to the first mask 102 on an inner surface of the first mask 102. The ADLF 112 is coupled to the first mask 102 adjacent a seal 110 (or frame) configured to inhibit any light from escaping about the outer edges of the ADLF 112. The seal 110 may be made of plastic and/or rubber, or another suitable material for creating a seal between the ADLF 112 and the first mask 102. The ADLF 112 is further coupled to the first mask 102 by a frame 114 configured to seat the ADLF 112 within the inner face of the first mask 102. For example, the seal 110 and the frame 114, when placed together, may serve as a frame for the ADLF 112 that surrounds the edges of the ADLF 112.

One or more snaps 116 may couple and rigidity secure the frame 114 holding the ADLF 112 to the first mask 102. The frame 114 in conjunction with the seal 110 provides a snug and secured fit between the ADLF 112 and the first mask 102. In embodiments, the first mask 102 further defines a seat on the inside of the first mask 102 (not explicitly shown) to accept and secure the seal 110, ADLF 112, and the frame 114. In other embodiments, the frame 114 may secure the ADLF 112 to the first mask 102 via a friction fit or mechanical fastener other than the snaps 116. The snaps 116 shown in FIG. 2 to secure the frame 114 and the ADLF 112 to the first mask 102, are just one example of a fastener for securing the ADLF and the frame 114 to the first mask 102 and is not intended to limit the scope of this disclosure.

The seal 110, the ADLF 112, and/or the frame 114 are configured to be removed from the first mask 102 so as to enable a user to exchange the ADLF 112 for another ADLF (not shown) or an auto-darkening filter (ADF) for non-laser welding operations. Such removable assembly of the seal 110, ADLF 112, and/or the frame 114 provides advantageous flexibility and adaptability for users to only need one welding helmet for multiple welding systems, e.g., different laser welding systems operating at different light wavelengths or for MIG or TIG welding systems (which also emit radiation at different varying wavelengths).

The ADLF 112 is configured to cover or fill the window or opening 103 of the first mask 102. The seal 110 limits or inhibits any light from escaping around the ADLF 112 to reach the user's face. Thus, any light transmitted into the window or opening 103 is directed to the ADLF 112 or an ADF coupled to or installed in the first mask 102.

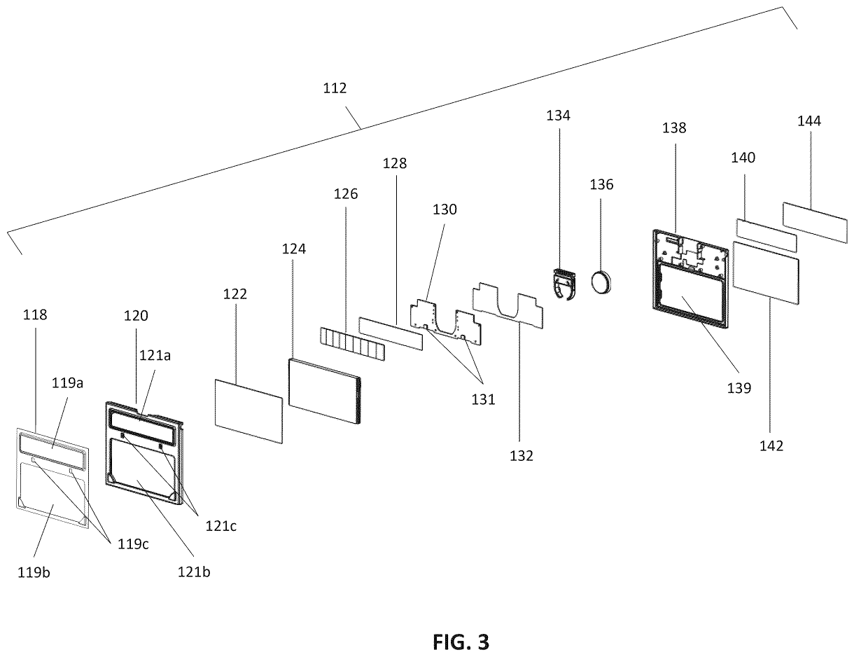

FIG. 3 illustrates an exploded view of the ADLF 112 of FIG. 2 according to embodiments of the present disclosure. The embodiment of the ADLF 112 shown in FIG. 3 is for illustration only. Other embodiments of the ADLF 112 could be used without departing from the scope of the present disclosure.

As illustrated in FIG. 3, the ADLF 112 comprises a metal panel 118, front housing 120, a laser lens 122, an auto-darkening filter (ADF) 124, a solar cell 126, a first metal film 128, a first circuit board 130 (e.g., a printed circuit board), a second metal film 132, a battery 136, a back housing 138, a second circuit board 140, an inner cover lens 142, and a control panel 144. A battery holder 134 can receive the battery 136 and maintain the battery 136 within back housing 138. In some embodiments, the battery 136 may be disposed outside the ADLF 112 but is in electrical communication with the ADLF 112.

The metal panel 118 is configured to define a first aperture 119a and a second aperture 119b, such that light may travel through the first and second apertures 119a, 119b, but not through the material of the metal panel 118. In various embodiments, the metal panel 118 is made from a metal, including aluminum and aluminum alloys, other lightweight metals, such as titanium. The metal panel 118 further includes at least one sensor aperture 119c, e.g., the two sensor apertures 119c shown in FIG. 3. The front housing 120 also has a first aperture 121a, a second aperture 121b, and at least one sensor aperture 121c. When the metal panel 118 and the front housing 120 are assembled together, the first and second apertures 119a, 119b, and the sensor apertures 119c of the metal panel 118 are in overlapping alignment with the first and second apertures 121a, 121b, and the sensor apertures 121c, respectively.

The solar cell 126 is configured to be seated within, the first aperture 119a and the first aperture 121a. The solar cell 126 is configured to harness light, including intense light emitted by welding, and convert the light energy into electricity. In some embodiments, the solar cell 126 provides power to the first and second circuit boards 130, 140, and/or the ADF 124. The solar cell 126 is in electric communication with the battery 136 and is configured to convert light into electrical energy. In some embodiments, the solar cell 126 is configured to charge the battery 136. To limit or inhibit exposure to harmful light passing through the solar cell 126 to the wearer of the welding helmet 100, the first metal film 128 is applied to or positioned behind the solar cell 126. The first metal film 128 absorbs or reflects the light transmitted to it. The first metal film 128 may be made from a metal, including aluminum and aluminum alloys, other lightweight metals, such as titanium.

The first circuit board 130 is positioned behind the solar cell 126 and the first metal film 128 and aligned such that one or more sensors 131 disposed on the circuit board, or in electrical communication with the first circuit board 130, are in overlapping alignment with one sensor aperture 119c and the respective sensor aperture 121c. In other words, one sensor 131 is provided for each respective sensor aperture 119c and 121c. For example, if four sensor apertures 119c and four respective sensor apertures 121c are present, then four sensors 131 are provided. To further inhibit or limit any harmful light from passing through the sensors 131 and the first circuit board 130 to the wearer of the welding helmet 100, the second metal film 132 is applied to the inner face of the first circuit board 130. The second metal film 132 may be made from a metal, including aluminum and aluminum alloys, other lightweight metals, such as titanium.

The sensors 131 may be optical sensor configured to sense an arc light (e.g., an amount of light meeting or exceeding one or more predefined or programmable thresholds), the light emitted from the welding laser, or the light emitted from the material being welded as the material heats up and melts. Upon receiving a signal from the sensors 131, control circuitry (e.g., one or more processors or microprocessors) on the first circuit board 130 or the second circuit board 140 may send a signal to control the ADLF 112 to transition from a transparent state (e.g., such as for greater visibility during grinding operations or prior to welding) to an opaque state to significantly limit the amount of light passing through the ADLF 112 such as for welding operations.

The first circuit board 130 may be configured to receive the battery holder 134. The battery holder 134 is configured to receive the battery 136. The battery 136 provides power to at least the first circuit board 130, including the one or more sensors 131. In some embodiments the battery 136 further provides power to the second circuit board 140 and the control panel 144. The first and second circuit boards 130 and 140 are in electrical communication with each other.

The second circuit board 140 is disposed on the inner face of the back housing 138. The control panel 144 is disposed on the second circuit board 140. The inner cover lens 142 is configured to cover an aperture 139 of the back housing 138. The inner cover lens 142 is configured to protect the ADF 124 and the laser lens 122. The inner cover lens 142 is further configured to transmit any light passed through the ADF 124. In embodiments, if the inner cover lens 142 becomes scratched, foggy, or otherwise damaged, the inner cover lens 142 may be removed and replaced with new inner cover lens 142.

At least one of the first circuit board 130 or the second circuit board 140 includes a memory and a processor in communication with the memory. The memory may be configured to store instructions executable by the processor. The processor may be, for example, a microprogrammed control unit.

The laser lens 122 and the ADF 124 are each configured to cover the second aperture 119b and the second aperture 121b. The laser lens 122 may comprise a glass optical filter. The glass optical filter, in various examples, may be a notch filter, a longpass filter, a shortpass filter, a hot mirror filter, a neutral density filter, and/or an absorptive or reflective glass optical filter. Any glass optical filter, or combination of class optical filters, configured to inhibit transmission of a wavelength of light emitted by the welding laser (or as of an otherwise predetermined wavelength) may be used to comprise the laser lens 122.

The laser lens 122 is configured to specifically filter out the wavelengths of light in which the welding laser operates. For example, in certain embodiments, the laser lens 122 may be configured to filter out light from about 1060 nanometers (nm) to about 1090 nm light wavelengths, or 1,060 nm to 1,068 nm. In other examples, the laser lens 122 may be configured to filter out light from about 1,064 nm to about 1,080 nm. In other examples, the laser lens 122 may be configured to filter wavelengths from 1064 nm to 1070 nm. In other aspects, the laser lens may filter wavelengths from about 1070 nm to about 1080 nm.

Generally, laser welding is most effective in the wavelengths around 1,060 nm to 1,080 nm. In embodiments, the laser lens may filter wavelengths at about 10,600 nm emitted by a carbon dioxide laser welding system, a wavelength that is also found to be effective for some welding applications. However, any suitable filter wavelength or range of wavelengths may be used for the laser lens 122 as desired by the specific type of laser used for laser welding operations.

Various embodiments of the present disclosure recognize that certain auto darkening filters may not specifically filter out such wavelengths because those wavelengths were not the strongest emitted wavelengths produced by traditional welding methods like MIG or TIG welding. Various embodiments of the present disclosure also recognize that welding auto darkening filters may not specifically filter out such wavelengths because traditional welding methods like MIG or TIG welding produced ultraviolet (UV) or infrared (IR) light in many more wavelengths. Accordingly, various embodiments of the present disclosure provide for the laser lens 122 to filter the relevent wavelengths for any laser welding system, including smaller wavelength laser welding equipment. The above examples are not intended, and should not be construed, to limit the present disclosure to the provided wavelengths of light to the exclusion of other wavelengths of light emitted by a given laser welding device.

The ADF 124 is positioned behind the laser lens 122 and is configured to cover the laser lens 122. The back housing 138 is configured to couple to the front housing 120 as to contain the laser lens 122, the ADF 124, the solar cell 126, the first metal film 128, the first circuit board 130 (e.g., a printed circuit board), the second metal film 132, and the battery 136.

The ADF 124 further inhibits harmful radiation in the infrared and ultraviolet light spectrums from passing through to a user's face. For example, the ADF 124 may be a ultraviolet/infrared liquid crystal display (UV/IR LCD) that filters ultraviolet and infrared light and includes an LCD to filter visible light. In various embodiments, the ADF 124 includes several layers (e.g., at least two layers) of liquid crystal cells placed between three polarization filters. At least one polarization filter is oriented at 90 degrees relative to the other polarization filter, such that, when the ADF 124 is not powered on, a default shade level is provided, (e.g., shade level 5). When powered, the ADF 124 may provide a default powered on state of shade level 3. When the sensors 131 detect light emitted from welding, the ADF 124 automatically darkens to a preset shade (e.g., shade 11 or shade 13). For example, a user may set the ADLF 112 to darken to shade 9 upon detection of light emitted from welding by sensors 131.

The ADF 124 transitions from one shade to another based on the orientation of the liquid crystals in the LCD. For example, in a first state defining a first shade, the liquid crystals are randomly oriented, allowing light to pass through. By passing an electric current through the liquid crystals, the crystals are aligned in a second state defining a second shade wherein the liquid crystals are oriented in a specified direction to bend any light incident on the liquid crystals. In other words, the polarization of the light passing through the ADF 124 may be altered. The direction may be specified based on the current passed through the ADF 124. Each layer is individually controlled, such that in one layer the liquid crystals bend light in one direction, and in another layer the liquid crystals bend light in a different direction. By controlling the orientation of each layer of liquid crystals and the intensity of the electromagnetic radiation may be reduced or increased (i.e., darkened or lightened) and thus the shade level provided by the ADLF 112 is controlled.

The control panel 144 enables a user to specify the desired shade level provided by the ADLF 112. The user may control the sensitivity of the sensors 131, the shade levels, and/or a response time or delay parameter of the ADLF 112 to change shades. In some embodiments, the welding helmet 100 will have external controls (e.g., disposed on an outer surface of the first mask) for manipulating the desired shade levels. In aspects, the ADLF 112 may include a control panel 144 that includes a digital display unit for controlling the sensitivity of the sensors 131, the shade levels, and/or a response time or delay parameter of the ADLF 112. The control panel 144 may include a plurality of buttons or dials for setting the shade level, sensitivity level, or delay parameter.

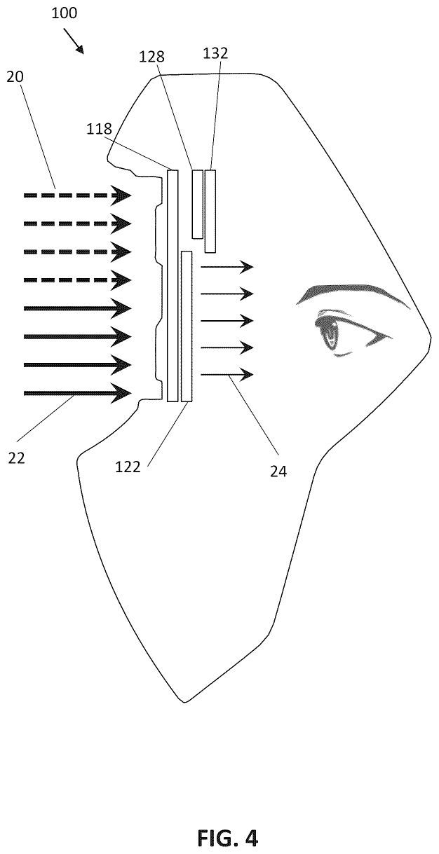

FIG. 4 illustrates an example diagram of the welding helmet 100 selectively filtering harmful light or radiation emitted towards a user's eye. In this example, FIG. 4 illustrates the arrangement and ordering of components included in the welding helmet 100 that limit or prevent laser light from reaching the user. As noted above, additional components may be included in the welding helmet 100 for similar or unrelated purposes. The embodiment of the light filtering shown in FIG. 4 is for illustration only. Other embodiments could be used without departing from the scope of the present disclosure.

In FIG. 4, the parallel lines with arrows illustrate beams of radiation and the radiation includes laser radiation 20 and other welding radiation 22. The radiation encounters in order, and is blocked by, the metal panel 118, the laser lens 122, the first metal film 128 and the second metal film 132. The metal panel 118 blocks or absorbs a first portion of the harmful radiation, while the laser lens 122 and the first and second metal films 128 and 132, respectively, further block the harmful radiation that is not absorbed or deflected by the metal panel 118.

As shown, the first metal film 128 does not cover the full extent of the second metal film 132 because, as explained above, the sensors 131 are disposed below the first metal film 128. The sensors 131 require light from the welding laser or system to activate the auto-darkening features of the ADLF 112 and thus should not be blocked by the first metal film 128.

In combination, the metal panel 118, the laser lens 122, and the first and second metal films 128, 132, respectively, filter out the harmful radiation while allowing in remaining unharmful light 24 (e.g., light in the visible spectrum). The unharmful light 24 may be darkened (or filtered) using the ADF 124.

In use, when a laser welding system is turned on, the light emitted by a laser is detected by the sensors 131. The sensors 131 send a signal to the first and second circuit boards 130 and 140, respectively, which causes a controller to pass a current through ADF 124 to change the opacity of the ADF 124 commensurate with a selected shade number. When the sensors 131 detect the end of a welding operation, that is, that the laser welding system is not welding a material, the sensors 131 send a signal to the first and second circuit boards 130 or the microprogrammed control unit (MCU) (not shown). The MCU may read a delay parameter, shade parameter, and sensitivity parameter, and, after receiving the signal from the sensor indicating the beginning or ending of a welding operation, output a corresponding electrical current, voltage, or signal to the ADF 124.

Although a laser welding system may not directly emit visible bright light, because a laser welding system significantly energizes the material being welded, bright visible light is emitted at the weld spot. Such light may be uncomfortable or damaging to the human eyes. The ADF 124 enables a user to darken or filter that remaining visible light.

In various embodiments, the welding helmet 100 protects against reflection, scattering, and diffraction from the laser light used in laser welding. For example, even if the laser beam does not directly shine on the user's face or eyes, when the laser beam encounters an object, reflection, scattering, diffraction, and divergence can occur. These phenomena cause the laser energy to disperse but can still potentially harm the user. Therefore, when using laser welding devices, in addition to preventing direct exposure to the face or eyes, the arrangement of the various protective layers of the welding helmet 100 assist to avoid or to reduce the potential risk of injury due to indirect exposure to laser light, for example, due to reflection, scattering, and diffraction of the laser beam.

In various embodiments, any combination of the second mask 104, the metal panel 118, the laser lens 122, the first metal film 128 and the second metal film 132 may further serve to absorb, resist, or disperse heat energy that is emitted from the laser beam during welding. For example, the second mask 104 and the metal panel 118 may absorb and conduct heat from the laser away from other the components of the welding mask 100, such as, those containing electronic circuitry, to prevent or reduce deformation or malfunction of those components due to the heat produced during laser welding.

One advantage of the welding helmet 100 is adaptability of the welding helmet for varying welding situations. The ADLF 112 may be easily removed from the inner face of the first mask 102 and replaced with another ADLF 112 configured for a different laser welding system, or for a different ADF for more standard welding techniques. To exchange the ADLF 112 for another ADF, a user can remove the snaps 116. In embodiments, the snaps 116, or other mechanical fastener that is easily removed, by hand without a tool or with a common or simple tool (e.g., a screwdriver). For example, a welder first using a laser welding system can install or if already installed, leave in place, the ADLF 112. When the user switches to a conventional welding system, e.g., a MIG welding system, the user can remove the ADLF 112 and insert a general purpose ADF. This enables a welder to only carry or purchase one helmet, rather than two or more helmets, and instead carry much lighter and smaller varying units of ADLFs 112 and general purpose ADFs (not shown).

Although the present disclosure has been described with exemplary embodiments, various changes and modifications may be suggested to one skilled in the art. It is intended that the present disclosure encompass such changes and modifications as fall within the scope of the appended claims. None of the descriptions in this application should be read as implying that any particular element, step, or function is an essential element that must be included in the claims scope. The scope of patented subject matter is defined by the claims.

What is claimed is:

1. A welding helmet comprising:
a first mask having an opening;
a second mask having an opening corresponding to the opening of the first mask, wherein the second mask is positioned on the first mask such that the second mask at least partially follows a profile of the first mask; and
an auto-darkening laser filter positioned adjacent to the opening of the first mask, the auto-darkening laser filter comprising:
a laser lens configured to filter a range of wavelengths of light, the laser lens positioned on the auto-darkening filter, wherein the range of wavelengths includes wavelengths from 1060 nanometers (nms) to about 1090 nms;
an auto-darkening filter positioned adjacent to the laser lens and configured to selectively filter light, wherein the auto-darkening filter is configured to provide a plurality of selectable shade levels;
a solar cell configured to generate electrical energy for the auto-darkening laser filter; and
a battery configured to provide power to the auto-darkening laser filter, wherein the solar cell is in electrical communication with the battery and is configured to charge the battery;
a cover lens positioned on an exterior surface of the second mask adjacent to the opening, wherein the cover lens is removable and replaceable;
an inner cover lens positioned adjacent to the auto-darkening laser filter, wherein the inner cover lens is configured to protect the auto-darkening filter and the laser lens and is removable and replaceable; and
a control panel configured to enable a user to select among the plurality of shade levels, wherein the control panel comprises a digital display unit.

2. The welding helmet of claim 1, wherein the auto-darkening laser filter further includes:
a sensor positioned adjacent to a second aperture of a panel of the auto-darkening laser filter, the sensor configured to generate a signal based on sensed light to control an operation of the auto-darkening filter, and
a metal film positioned behind the sensor and the second aperture.

3. The welding helmet of claim 2, wherein:
the metal film is a first metal film; and
the auto-darkening laser filter further includes a second metal film positioned behind the solar cell.

4. The welding helmet of claim 3, wherein the second mask, the panel, the laser lens, the first metal film, and the second metal film are positioned relative to each other such that light radiating toward eyes of a wearer of the welding helmet, when worn, is inhibited or filtered by at least one of the second mask, the panel, the laser lens, the first metal film, and the second metal film.

5. The welding helmet of claim 1, wherein:
the first mask comprises a non-metallic material, and
the auto-darkening laser filter is secured within the openings of the second and first mask.

6. The welding helmet of claim 5, wherein the second mask and the auto-darkening laser filter are removable from the first mask.

7. The welding helmet of claim 5, further comprising:
a frame surrounding an outer surface of the auto-darkening laser filter,
wherein the frame including the auto-darkening laser filter is coupled to the first mask via at least one snap.

8. The welding helmet of claim 7, wherein:
the frame includes at least a portion including a rubber material, and
the rubber material is directly adjacent to the first mask when the frame is coupled to the first mask.

9. The welding helmet of claim 1, wherein:
the welding helmet is a laser welding helmet configured to filter or limit exposure to laser light; and
the range of wavelengths of light filtered by the laser lens is associated with the laser light.

10. The laser welding helmet of claim 9, wherein the range of wavelengths of light is from about 1060 nms to about 1080 nms.

11. The welding helmet of claim 1, wherein the auto-darkening laser filter further comprises a panel comprising an aperture, the panel positioned on a surface of the auto-darkening laser filter and adjacent to the second mask.

12. The welding helmet of claim 1, wherein the auto-darkening laser filter is removable from the first mask.

13. The welding helmet of claim 1, wherein the auto-darkening laser filter further comprises:
a panel defining a first aperture, a second aperture, and at least one sensor aperture,
wherein the panel is positioned on a surface of the auto-darkening laser filter.

14. The welding helmet of claim 13, wherein:
the solar cell is seated within the first aperture; and
the laser lens and the auto-darkening filter are positioned behind the second aperture.

15. The welding helmet of claim 14, further comprising:
a first metal film positioned behind the solar cell and the first aperture, wherein the first metal film is configured to absorb or reflect light transmitted through the solar cell.

16. The welding helmet of claim 15, further comprising:
a sensor aligned with the at least one sensor aperture, the sensor configured to detect light emitted during welding operations; and
a second metal film positioned behind the sensor and the at least one sensor aperture.

17. An apparatus for filtering light, the apparatus comprising:
a laser lens configured to filter a range of wavelengths of light associated with laser welding, wherein the range of wavelengths includes wavelengths from about 1060 nanometers (nms) to about 1090 nms;
an auto-darkening filter positioned adjacent to the laser lens and configured to selectively filter light, wherein the auto-darkening filter is configured to provide a plurality of selectable shade levels;
a solar cell configured to generate electrical energy for the apparatus;
a battery configured to provide power to the apparatus, wherein the solar cell is in electrical communication with the battery and is configured to charge the battery;
a cover lens positioned on an exterior surface of the apparatus, wherein the cover lens is removable and replaceable;
an inner cover lens positioned adjacent to the auto-darkening filter, wherein the inner cover lens is configured to protect the auto-darkening filter and the laser lens and is removable and replaceable; and
a control panel configured to enable a user to select among the plurality of shade levels, wherein the control panel comprises a digital display unit.

18. The apparatus of claim 17, wherein the apparatus is removable from a welding helmet.

19. The apparatus of claim 17, wherein:
the apparatus is configured to filter or limit exposure to laser light; and
the range of wavelengths of light filtered by the laser lens is associated with the laser light.

20. The apparatus of claim 19, wherein the range of wavelengths of light is from about 1060 nms to about 1080 nms.
